(19) United States
(12) Patent Application Publication
(10) Pub. No.: US 2022/0195475 A1
(43) Pub. Date: Jun. 23, 2022

(54) HYDROLYSIS OF BREWER'S SPENT GRAIN

(71) Applicant: HEALLO S.R.L., Milano (IT)

(72) Inventor: Francesca Varvello, Milano (IT)

(73) Assignee: HEALLO S.R.L., Milano (IT)

(21) Appl. No.: 17/602,747

(22) PCT Filed: Apr. 7, 2020
(86) PCT No.: PCT/IB2020/053299

§ 371 Date: Oct. 9, 2021

(30) Foreign Application Priority Data
Apr. 11, 2019 (IT) 102019000005588

(51) Int. Cl. C12P 19/14; C12P 19/04; C12P 7/42; C07K 14/415; C12C 7/04; C12C 7/20; C12C 11/00; C12C 11/11; C12C 7/16; C12C 12/00; A23L 2/52; A23G 1/40; A23G 1/48

(52) U.S. Cl. CPC C12P 19/14; C12P 19/04; C12P 7/42; C07K 14/415; C12C 7/04; C12C 7/205; A23V 2002/00; C12C 11/11; C12C 7/16; C12C 12/00; A23L 2/52; A23G 1/40; A23G 1/48; C12C 11/003

(57) ABSTRACT

A product hydrolysate from brewers' spent grain is disclosed, and the process for the preparation thereof, as well as uses thereof in food for humans and feed for animals. This hydrolysate contains a high percentage of pentosans at a low molecular weight which therefore offers a high bioavailability of soluble fibres. This allows the drawbacks associated with a high consumption of wholegrain products to be overcome, while also allowing consumers to benefit from the nutritional components contained therein.

### FIELD OF THE INVENTION

The present invention concerns a plant fibre hydrolysate product, in particular from brewers' spent grain, the process for the preparation thereof, as well as the uses thereof in food for humans and feed for animals.

### BACKGROUND ART

Food-grade plant fibre has been renowned for some time now as a dietary component which has the capacity to influence multiple aspects of the natural digestion process.

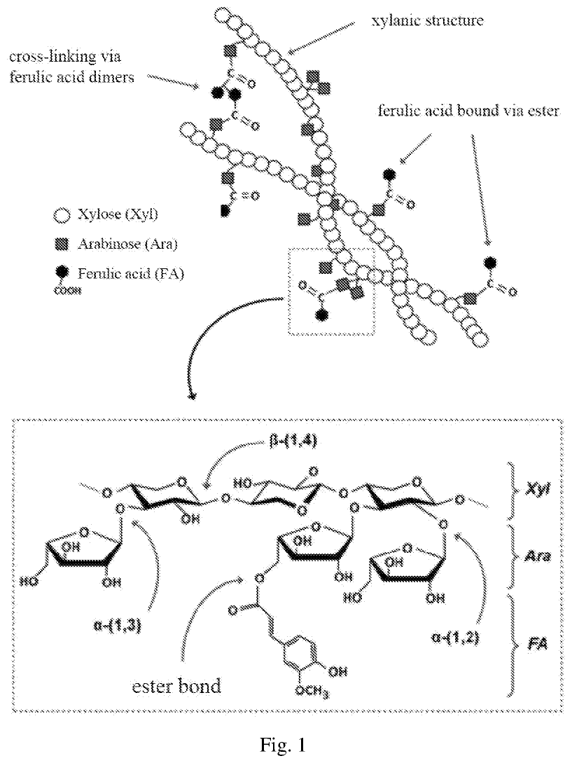

Indeed, it is known that the intake of bran and likewise of wholegrain cereals has a positive effect on intestinal regularity and on the prevention of certain diseases. It is only recently though that various studies have shown that these benefits are not attributable exclusively to the mechanical effect of “dragging and cleaning” which fibre-rich plants produce, but also and especially to clearly defined molecules which comprise these plant matrices. Food-grade plant fibre consists of insoluble fibre, i.e. from cellulose and lignin, which acts prevalently on gastrointestinal tract function, promoting the dragging and cleaning effect mentioned above, because it promotes transit of the bolus in the intestine and evacuation of the faeces. But food-grade plant fibre is also composed of soluble fibre, primarily constituted of polysaccharidic chains of arabinoxylans belonging to the family of pentosans and ferulic acid, an antioxidising molecule associated with pentosanic structures, as shown in FIG. 1.

These soluble fibres are also present in what is known as ‘brewers' spent grain’, i.e. a by-product of the brewing industry. Brewers' spent grain consists of the residue from the hot extraction of malted grain: it comprises the outer husks of the grain and the fractions which have not undergone solubilisation in the malting and mashing process, and likewise variable amounts of non-saccharificated starch and dextrin.

In the production of beer, apart from malted barley, other—non-malted—grains are also used.

Fresh brewers' spent grain, however, has an analytical composition which is quite constant, despite the differences in the grains employed, in other words, in terms of dry weight, they typically comprise:

protein: approximately 30%
complex carbohydrates: approximately 50%, including 22-23% arabinoxylans, 12-25% cellulose, and 10% beta-glucans

The composition is such that it is possible to attribute the spent grain a nutritional value of above 0.80 U.F.L. and 0.75 U.F.C. per kg dry matter, a value which is not far from that of wheat bran (U.F.L.=Unitá Foraggera Latte i.e. milk fodder unit, which is equivalent—in terms of nutritional power—to 1 kg barley grain which, when administered to lactating cows, provides approximately 1700 kcal and allows the production of 2.33 l of milk with a 4% fat content. U.F.C.=Unitá Foraggera Carne; i.e. met fodder unit, which is equivalent—in terms of nutritional power—to 1 kg of barley grain which provides 1820 kcal for the maintenance and growth of animals in the fattening condition). Given this nutritional value, brewers' spent grain is employed in particular in feeding farm animals.

Pentosans, and in particular arabinoxylans, regulate the absorption of sugars and fats contributing to the control of the level of glucose and cholesterol in the blood. They therefore play an active role in reducing glycaemia and in controlling the hypercholesterolemia and obesity.

Furthermore, arabinoxylans are known prebiotics which are able to increase the faecal bifidobacterial and reduce the urinary excretion of p-Cresol, improving intestinal health and the immune system overall.

However, almost all the pentosans (including arabinoxylans) and the ferulic acid present in food-grade plant fibre in general, are not bioavailable as they are strictly connected to other inert structures such as cellulose and lignin.

The very incapacity of the digestive tract to break down these structures has led the probiotic intestinal microflora in the large intestine to produce hydrolytic enzymes which, in the presence of food-grade fibre, separate pentosans and ferulic acid from the compounds to which they are bound, making them bioavailable. However, this process is slow and, as it only occurs in the final tract of the intestine, has much lower yields.

Although the positive effects of the intake of plant fibres have been established, it must be noted that the intake of an amount consisting of 8 g arabinoxylan-rich fibre in 100 g of carbohydrates can lead to a series of disorders, such as, for example, bloating, meteorism, and irritation of the colon and ensuing abdominal pains, due to the part consisting of insoluble fibre.

Furthermore, the wholegrain foods which must be consumed in order to reach an arabinoxylan-rich fibre content amounting to 8 g out of 100 g are, in general, less appetising and contain lipase inhibitors, which render the pancreatic lipases ineffective, and phytates (which are indigestible to humans or non-ruminant animals and are classified as anti-nutritional due to the chelating effect they have on certain nutritional elements). Indeed, if consumed in large amounts, phytates inhibit the metabolism and the absorption of numerous minerals, such as calcium and zinc, as well as vitamin B1, rendering some proteins indigestible.

A further consideration which should be made is that the insoluble part of the food-grade plant fibre may be contaminated by mycotoxins, which alter the immune and neurological systems, cause oxidative stress, and damage the intestinal barrier.

It is therefore object of the present invention to provide a product with high bioavailability of the soluble fibres mentioned earlier, which also reduces as greatly as possible the disadvantages associated with a high level of consumption of known wholegrain products.

### SUMMARY OF THE INVENTION

Said object has been achieved by a process for the preparation of a hydrolysate from brewers' spent grain, as stated in claim 1.

In a further aspect, the present invention concerns hydrolysates from brewers' spent grain obtainable by this process.

In a still further aspect, the present invention concerns food compositions, food supplements, and food products comprising said hydrolysates from brewers' spent grain.

In another aspect, the present invention concerns a brewing process which envisages the use of these hydrolysates.

In another aspect, the present invention concerns a process for the preparation of a phytocomplex which envisages the use of these hydrolysates.

The characteristics and the advantages of the present invention will become apparent from the following detailed description and the working examples provided for illustrative and non-limiting purposes.

### DETAILED DESCRIPTION OF THE INVENTION

The invention concerns, therefore, a process for the preparation of a hydrolysate from brewers' spent grain, said process comprising the steps of:
i) mixing brewers' spent grain and water, in a weight ratio of 1:1 to 1:5,
ii) adding to the mixture thus obtained up to 2 wt %, based on the weight of the mixture, an enzyme consisting of:
   a) xylanase, or
   b) an enzymatic complex of xylanase, amylase and glucanase,
and leaving to react for 1-6 hours at a temperature of 45-65° C. and a pH of 4-6,
iii) deactivating the enzyme of step ii), by increasing the temperature to 80-90° C. for at least 5 minutes, and
iv) separating the liquid component from the solid component obtained at the end of step iii), thus keeping the liquid component, which is the brewers' spent grain hydrolysate.

As will be seen hereinbelow, this process not only results in a hydrolysate which is advantageously enriched with pentosans with a medium and low molecular weight, therefore an enriched hydrolysate in the soluble fraction of the spent grain, but also makes it possible to reuse that which normally constitutes brewing production waste, in other words said spent grain.

The solid component remaining after step iv), in other words the spent grain which contains mostly cellulose and lignin, can be advantageously allocated to agriculture, either as such or, in case, in a granular or pellet form. Alternatively, the spent grain can find advantageous application as a substrate for the growth of microorganisms, e.g. of the genus Trichoderma, which are useful in agriculture, e.g. in the protection of what is known as pruning wounds, or as anti-phytopathogenic agents.

The process of the invention is therefore particularly advantageous as it employs a form of production waste as its raw material and all the materials resulting therefrom have an intended use, thereby eliminating waste overall.

Preferably, said xylanase is endo-1,4-beta-xylanase.

Preferably, said amylase is alpha-amylase.

Preferably, said glucanase is endo-1,3(4)-beta-glucanase.

In some preferred embodiments, in step i), brewers' spent grain and water are in a weight ratio of 1:1.5 to 1:3.

In other preferred embodiments, in step ii), the enzyme is added in amounts up to 1 wt %, based on the weight of the mixture. In more preferable embodiments, in step ii), the enzyme is added in amounts of 0.1-0.7 wt %, based on the weight of the mixture.

In further preferred embodiments, in step ii) the mixture is left to react for 2-4 hours at 58-62° C.

In step iii), the enzyme is deactivated by heat treatment, in other words by raising the temperature. Preferably, the deactivation occurs at approximately 85° C. for approximately 15 minutes.

In step iv), the separation of the liquid component can be performed by using known techniques of filtration, centrifugation or both. Optionally, further rinsing with water is also possible. The hydrolysate of the invention therefore offers, advantageously, a source of soluble fibre substantially devoid of lignin and cellulose, which therefore does not determine irritation phenomena in the colon and consequent meteorism and abdominal pain.

Optionally, the process of the invention further comprises a step v) wherein the liquid component resulting from step iv) is dried, thus obtaining a dry hydrolysate from brewers' spent grain. The drying step v) is preferably performed at temperatures not higher than 65° C. This drying step v) can be performed by lyophilisation.

In a further aspect, the present invention concerns a first hydrolysate from brewers' spent grain obtainable by the process described above, wherein in step ii) the enzyme xylanase a) is added, said hydrolysate comprising ferulic acid, proteins, up to 30 mg beta-glucans and up to 300 mg pentosans having a number average molecular weight not higher than 30 kDa per gram of dry hydrolysate.

The expression “desiccated hydrolysate” or “dry hydrolysate” means a hydrolysate which has undergone desiccation. A hydrolysate is deemed desiccated or dry if it features a residual water content not higher than 1 wt %, based on the weight of the dry hydrolysate.

Surprisingly, it was observed that the presence of the enzyme xylanase, under the process conditions stated above, allows to release the more soluble fractions from the spent grain, such as the pentosans with low and medium molecular weight, as well as beta-glucans, ferulic acid, and proteins.

Preferably, the hydrolysate from brewers' spent grain comprises up to 25 mg beta-glucans and up to 250 mg pentosans having a number average molecular weight not higher than 30 kDa per gram of dry hydrolysate.

Preferably, the hydrolysate from brewers' spent grain comprises up to 0.5 mg ferulic acid per gram of dry hydrolysate.

Preferably, the hydrolysate from brewers' spent grain comprises up to 8 mg proteins per gram of dry hydrolysate.

In a further aspect, the present invention concerns a second hydrolysate from brewers' spent grain obtainable by the process described above, wherein during step ii) the enzymatic complex b) of xylanase, amylase, and glucanase is added, said hydrolysate comprising ferulic acid, proteins, traces of beta-glucans and up to 250 mg pentosans having a number average molecular weight not higher than 10 kDa per gram of dry hydrolysate.

Surprisingly, it was observed that the presence of xylanase as well as that of amylase and glucanase, under the process conditions stated above, allows to release the more soluble fractions from the spent grain, such as the pentosans with low molecular weight, as well as the ferulic acid and proteins.

Preferably, the hydrolysate from brewers' spent grain comprises up to 200 mg pentosans having a number average molecular weight not higher than 5 kDa per gram of dry hydrolysate.

Preferably, the hydrolysate from brewers' spent grain comprises up to 0.8 mg ferulic acid per gram of dry hydrolysate.

Preferably, the hydrolysate from brewers' spent grain comprises up to 5 mg proteins per gram of dry hydrolysate.

In some preferred embodiments, the hydrolysate from brewers' spent grain of the invention is a dry hydrolysate. The availability of a dry product offers a number of advantages, which range from the manageability of the volumes, which are smaller than those of the corresponding aqueous solutions, to the ensuing practical transportation, in addition to the storage life, which is due to the significant reduction in both the risk of bacterial contamination and the risk of rancidity. Naturally, should the circumstances require it, the hydrolysate could easily be solubilized in water to bring the product into a liquid form with the desired concentration.

The determination of the pentosans is based on a rapid reproducible phloroglucinol-based colorimetric method (S. G. Douglas, “A rapid method for the determination of pentosans in wheat flour”, Food Chemistry, 7, 1981, 139-145) by using D-xylose, at 510-552 nm, as a reference standard. Since the method envisages an acid hydrolysis at high temperatures, all the pentosans are counted regardless of the degree of polymerization thereof. For the purposes of the present invention, the number average molecular weight (Mn) of the pentosans in hydrolysate is measured by using diafiltration on sieves at different molecular cut-off points: 50 kDa, 30 kDa, 10 kDa and 5 kDa.

The ferulic acid is preferably quantified by reverse phase HPLC analysis on a column C18, using a gradient of acetonitrile in water and trifluoracetic acid.

As said ferulic acid is present in a form which is unbound from the structures of the plant fibre, it is more bioavailable and therefore more effective.

The process as stated above has shown how the selection of the chosen enzyme not only allows the active soluble molecules to be separated from the inactive insoluble matrix of the spent grain, but also the mean molecular dimensions of said active molecules to be reduced to a molecular dimensions which are easy to use and exploit inside the body. Indeed, the digestion of a molecular complex with dimensions in the order of a few microns is slower and more difficult than the digestion of molecules with dimensions in the order of a few kilodaltons (kDa).

In a further aspect, the present invention also concerns a food composition comprising said first hydrolysate from brewers' spent grain and said second hydrolysate from brewers' spent grain. Indeed, since depending on the enzyme employed, the resulting hydrolysate features active components at different concentrations, it would be advantageous to combine different hydrolysates depending on the different nutritional needs.

In a further aspect, the present invention concerns a food product comprising said first hydrolysate from brewers' spent grain, or said second hydrolysate from brewers' spent grain, or the food composition comprising both, said food product being an edible product selected from baked goods, feeds, food supplements, nutraceuticals, alcoholic drinks, soft drinks, energy drinks, dietary bars, food oils, breakfast cereals, fresh pasta, dry pasta, yoghurt, ice creams, fruit juices, and confectionery, such as chocolate.

Preferably said food product is beer.

In a further aspect, the present invention concerns a food supplement for humans and/or animals, comprising said first hydrolysate from brewers' spent grain, or said second hydrolysate from brewers' spent grain, or the food composition comprising both.

In a still further aspect, the present invention concerns a medical device for humans and/or animals, comprising said first hydrolysate from brewers' spent grain, or said second hydrolysate from brewers' spent grain, or the food composition comprising both. Examples of medical devices are molecular complexes whose action is due to the polysaccharidic component of arabinoxylans (molecular weight>20,000 Dalton), endowed with a high affinity for mucosae, whose viscous properties allow the formation of a barrier-effect which reduces and modulates the absorption of sugars and reduces postprandial glycaemic spikes.

In some embodiments, said first hydrolysate from brewers' spent grain of the invention consists essentially of ferulic acid, proteins, up to 30 mg beta-glucans and up to 300 mg pentosans having a number average molecular weight not higher than 30 kDa per gram of dry hydrolysate. The expression “consists essentially of” means that ferulic acid, proteins, beta-glucans and pentosans having a number average molecular weight not higher than 30 kDa are the sole active ingredients present in the hydrolysate, while further components or excipients do not interfere with the action thereof. It should be understood that all the aspects identified above as preferred and advantageous for the hydrolysate and the components thereof should likewise be deemed preferred and advantageous also for these embodiments.

In other embodiments, said first hydrolysate from brewers' spent grain of the invention consists of ferulic acid, proteins, up to 30 mg beta-glucans and up to 300 mg pentosans having a number average molecular weight not higher than 30 kDa per gram of dry hydrolysate, and optionally water.

It should be understood that all the aspects identified hereinabove deemed preferred and advantageous for the hydrolysate and the components thereof, should likewise be deemed to be preferred and advantageous also for these embodiments.

In some embodiments, said second hydrolysate from brewers' spent grain of the invention consists essentially of ferulic acid, proteins, and up to 250 mg pentosans having a number average molecular weight not higher than 10 kDa per gram of dry hydrolysate. The expression “consists essentially of” means that ferulic acid, proteins, and pentosans having a number average molecular weight not higher than 10 kDa are the sole active ingredients present in the hydrolysate, while any further components or excipients do not interfere with the action thereof. It should be understood that all the aspects identified above as preferred and advantageous for the hydrolysate and the components thereof, should likewise be deemed preferred and advantageous also for these embodiments.

In other embodiments, said first hydrolysate from brewers' spent grain of the invention consists of ferulic acid, proteins, and up to 250 mg pentosans having a number average molecular weight not higher than 10 kDa per gram of dry hydrolysate and optionally water.

It should be understood that all the aspects identified above as preferred and advantageous for the hydrolysate and the components thereof should likewise be deemed preferred and advantageous also for these embodiments.

In a further aspect, the present invention concerns a brewing process which envisages the use of the hydrolysates described above.

Beer is a beverage traditionally made with 4 ingredients, i.e. water, malted barley, hops, and yeast.

The malted barley is obtained by making the grains of barley germinate and then interrupting the germination by drying. In this step, the grains of barley undergo important modifications and numerous substances which are essential for making beer are produced.

The hop plant is a climbing plant which grows in temperate zones. For brewing, the flowers are used, which give the beer bitterness and flavours and are a natural preservative.

Yeast is a single-cell organism which is responsible for the fermentation and principally produces, during the activity thereof, alcohol and carbon dioxide.

The process typically envisages the following main steps:
the malted barley is chopped and heated in water in a mashing tun, to promote the transformation of the starch in the barley into sugars,
when all the starches have been transformed into sugars, the wort is separated from the solid component by means of filtration (principally the husks of the barley grains),
the wort is transferred into a boiling tank and brought to boiling point with the hops, from which it acquires the characteristic flavours and bitterness,
the wort is then filtered to separate the same from the hops, and then cooled to a suitable temperature for fermentation,
the wort is transferred to a fermentation tank together with the yeast, typically Saccharomyces Cerevisiae,
at the end of the fermentation, the yeast on the bottom of the tank is collected and the beer thus obtained is transferred to the maturation room.

Finally, the beer is left to mature and then served, bottled, or barrelled.

The consumption of beverages with a low alcohol content, such as beer, is widespread and (in moderate amounts, naturally) has beneficial effects on glucose tolerance. However, beer is considered to be a high glycaemic index (or, for brevity, ‘GI’) food, in other words with a GI of approximately 110, and therefore not advisable in the diets of people who suffer from diabetes mellitus or glucose intolerance.

Actually, the GI of beer varies, firstly since the index is typically determined by using beverages with a variable glucose content as a reference standard, such as milk or orange juice, and secondly because there are different types of beer, i.e. craft beers and industrially brewed beers, and therefore the value stated is essentially an average.

In any case, since the values are nevertheless high, it is desirable to reduce the GI of beer. On this note, it has surprisingly been found that by employing the hydrolysates of the present invention in the brewing process, a low glycaemic index beer is obtained.

The brewing process of the present invention comprises the steps of:
1) adding water and the hydrolysate from brewers' spent grain obtained in step iv) of the process for the preparation of a hydrolysate from brewers' spent grain described above or the food composition described above, in a mash tun,
2) adding ground malt, thus proceeding to the mashing,
3) filtering the wort resulting from step 2),
4) boiling the wort and adding the hops,
5) separating the wort from the hops and fermenting in the presence of yeasts,
6) separating the yeasts and the solid residues from the beer thus obtained, and, optionally,
7) leaving the beer to age.

Preferably, the malt in step 2) is added in amounts of 10 to 30 wt %, based on the weight of the aqueous mixture resulting from step 1), more preferably in amounts of 15 to 20 wt %.

Preferably, the yeast employed in step 5) is Saccharomyces Cerevisiae.

Advantageously, the beer obtainable by the process described above has a lower glycaemic index than the same beer obtained according to conventional methods. Furthermore, the beer obtainable by the process described above preferably comprises 1.8-2.7 mg/ml pentosans in total, including 0.08-0.1 mg/ml xylose and 0.15-0.25 mg/ml arabinose, and comprises 1.5-2.0 ppm of ferulic acid, while said beer obtained by conventional methods typically comprises 0.75 mg/ml pentosans in total, including 0.0 mg/ml xylose and 0.15 mg/ml arabinose.

In a further aspect, the present invention concerns a phytocomplex preparation process which envisages the use of the hydrolysates described above, as well as phytocomplexes thus obtained.

In particular, the process for the preparation of a phytocomplex comprises the following steps:
A) preparing a fermentation broth starting from said first hydrolysate from brewers' spent grain, or from said second hydrolysate from brewers' spent grain, or from the food composition comprising both,
B) stabilizing the fermentation broth at a pH of 3-6.5,
C) inoculating the fermentation broth with a first mother culture of at least one microorganism selected from Komagataeibacter xylinus, Komagataeibacter swingsii, Komagataeibacter rhaeticus, and mixtures thereof, or with a second mother culture of at least one microorganism selected from Streptococcus thermophilus, Lactobacillus debruecki bulgaricus, Lactobacillus helveticus, Lactobacillus plantarum, Lactobacillus casei, and mixtures thereof,
D) leaving the broth to ferment,
E) inactivating the fermented broth, and
F) purifying the inactivated fermented broth to obtain a phytocomplex.

Preferably, the preparation of the culture broth in step B) involves keeping certain parameters within certain ranges, in particular:
pH is 3.0-6.5, preferably 3.0-4.3, more preferably approximately 3.5;
the concentration of the dissolved solids measured in degrees Brix is 8-25, preferably 13-20, more preferably approximately 15;
the temperature is 20-45° C., preferably 24-28° C., more preferably approximately 27° C.;
the concentration of the dissolved oxygen is 8-40 mg/l, preferably 12-20 mg/l, more preferably approximately 16 mg/l.

In step D) the fermentation broth is inoculated with at least one microorganism, which constitutes what is known as a fermentation starter. Preferably, said microorganism in the first mother culture is Komagataeibacter xylinus; preferably, said microorganism in the second mother culture is Streptococcus thermophilus, or Lactobacillus debruecki bulgaricus.

The amount of microorganism inoculated is 10²-10⁹ UFC/ml, preferably 10⁴-10⁷ UFC/ml, more preferably in the order of 10⁴ UFC/ml, with a inoculation dose of 0.1-20 wt %, preferably 0.2-5 wt %, more preferably 0.3-1 wt %.

The fermentation step D) is preferably performed under the following conditions:
pH of 2.5-6.0, preferably 2.5-3.8, more preferably approximately 3.0;
temperature of 10-32° C., preferably 18-30° C., more preferably approximately 28° C.;
the concentration of the dissolved oxygen is 8-40 mg/l, preferably 12-20 mg/l, more preferably approximately 16 mg/l;

In preferred embodiments, the fermentation step D) is performed:
for a time of 5-240 hours, more preferably 5-160 hours, even more preferably 7-20 hours; and
until a bacterial load of 10³-10⁷ UFC/ml is reached, more preferably 10⁴-10⁶ UFC/ml, even more preferably in the order of 10⁵ UFC/ml.

The fact that the fermented culture broth obtainable at the end of step D) can be utilised as a fermentation starter in bakery products is appreciable and advantageous.

The fact that the inactivated fermented culture broth obtainable at the end of step E) can be utilised in prebiotic products to produce reinforced prebiotic products is furthermore appreciable and advantageous.

In a further aspect, the present invention concerns a phytocomplex obtainable by the process described above, for use as an intestinal regulator.

In a further aspect, the present invention concerns a phytocomplex obtainable by the process described above, for topical use as a healing agent.

In view of what described and discussed above, it is clear that the object of providing a product with high bioavailability of soluble fibres has been achieved, i.e. the hydrolysates from brewers' spent grain, allowing at the same time the reduction of the disadvantages associated with a high consumption of known wholegrain products.

Furthermore, the further significant object of reusing a brewery waste product has been reached, while obtaining materials which find different applications and therefore eliminate waste overall.

It should be understood that all the possible combinations of the preferred aspects of the process for the preparation of the hydrolysate, of the uses thereof, and of the products which contain it, as stated above, are likewise preferred.

It should be understood that all the aspects identified as preferred and advantageous for the hydrolysate and the components thereof, should likewise be deemed preferred and advantageous also for the preparation and the uses of said hydrolysate.

Below are working examples of the present invention provided for illustrative purposes.

### EXAMPLES

### Example 1

Two hydrolysates from spent grain were prepared according to the present invention, by setting the following process parameters:
i) spent grain and water were mixed, the spent grain being 40 wt %,
ii) the enzyme was added in amounts of 0.2 wt %, at a pH of 5, bringing the temperature to 60° C., for a time of 4 hours,
iii) the enzyme was then deactivated by heating to 85° C. for 15 minutes,
iv) the resulting hydrolysate was separated by filtration from the solid residues.

### Results:

| | | Example 1a | Example 1b |
|---|---|---|---|
| | Enzyme used => | endo-1,4-beta- | endo-1,4-beta- |
| | | xylanase | xylanase + |
| | | | alpha-amylase + |
| | | | endo-1.3(4)-beta- |
| | | | glucanase |
| TOTAL SOL. PENTOSANS [mg/ml] | phloroglucinol | 4.417 | 7.771 |
| including: | | | |
| XYLOSE [mg/ml] | enzyme kit | 0.205 | 0.610 |
| ARABINOSE [mg/ml] | enzyme kit | 0.015 | 1.686 |
| FREE FERULIC ACID [ppm] | HPLC | 1.47 | 45.41 |
| PROTEINS [ppm] | Bradford | 63.8 | 82.1 |
| BETA-GLUCANS [ppm] | enzyme kit | 245.6 | 0.0 |
| DRY RESIDUE [%] | gravimetric | 1.41 | 2.86 |
| WATER CONTENT [%] | by difference | 98.59 | 97.14 |

After a subsequent drying step v), the hydrolysates had the following composition:

| | | Example 1a | Example 1b |
|---|---|---|---|
| | Enzyme used => | endo-1.4- | endo-1.4-beta- |
| | | beta- | xylanase + |
| | | xylanase | alpha-amylase + |
| | | | endo-1.3(4)- |
| | | | beta-glucanase |
| TOTAL SOL. | phloroglucinol | 313.26 | 271.71 |
| PENTOSANS | | | |
| [mg/g] including: | | | |
| XYLOSE [mg/g] | enzyme kit | 14.54 | 21.33 |
| ARABINOSE [mg/g] | enzyme kit | 1.06 | 58.95 |
| FREE FERULIC | HPLC | 0.104 | 1.588 |
| ACID [mg/g] | | | |
| PROTEINS [mg/g] | Bradford | 4.524 | 2.871 |
| BETA-GLUCANS | enzyme kit | 17.418 | 0.00 |
| [mg/g] | | | |
| DRY RESIDUE [%] | gravimetric | 100.00 | 100.00 |
| WATER | by difference | | |
| CONTENT [%] | | | |

Distribution of the molecular weights of the arabinoxylans obtained also on dry hydrolysate:

The analysis of the distribution by filtration on cut-off membranes are the following:

| Pentosans: | | Example 1a | Example 1b |
|---|---|---|---|
| 10-30 | kDa | 17% | 9% |
| 5-10 | kDa | 29% | 30% |
| <5 | kDa | 13% | 30% |

### Example 2

### Brewing Process

1st Day—Pre-Mashing: Spent Grain Hydrolysis

489 kg spent grain from prior brewing and 1050 1 mains water (total mass: 1539 kg) were mixed.

### Mashing Tun:

slow agitation of the mixture for 10 minutes
addition of enzyme: endo-1,4-beta-xylanase: 2.5 kg (0.51% of spent grain)
temperature increase to 60° C. in 10 minutes
maintenance at 60° C. for 3 hours:

| | Sampling after: | 1 h | 2 h | 3 h |
|---|---|---|---|---|
| TOTAL SOL. PENTOSANS | phloroglucinol | 3.560 | 4.103 | 4.755 |
| [mg/ml] including: | | | | |
| XYLOSE [mg/ml] | enzyme kit | 0.098 | 0.154 | 0.162 |
| ARABINOSE [mg/ml] | enzyme kit | 0.524 | 0.575 | 0.612 |
| FREE FERULIC ACID [ppm] | HPLC | 3.31 | 3.35 | 3.16 |

temperature increase to 85° C. in 15 minutes: deactivation of the enzyme

### Transfer to Boiling Tank:

The entire mixture was transferred to the boiling tank and filtered.

### Filtration:

Filter mesh with 2 μm diameter openings. Spent grain was retained while the liquid containing the substance solubilised by the enzyme flowed through. To facilitate complete extraction a washing was performed by adding mains water from above (200 litres). Filtration occurred through slow falling (1 hl in 5 minutes) and then a blade agitator was activated, which generated a mild agitation action, promoting the percolation of the liquid from the brewers' spent grains.

### Hydrolysate was Returned to the Mashing Tun:

Hydrolysate was returned to the mashing tun: transfer of all the hydrolysate (12 hl in total) occurred in 60′ (temp: approximately 70° C.):

Sampling:

| | | |
|---|---|---|
| TOTAL SOL. PENTOSANS [mg/ml] | phloroglucinol | 3.321 |
| including: | | |
| XYLOSE [mg/ml] | enzyme kit | 0.059 |
| ARABINOSE [mg/ml] | enzyme kit | 0.331 |
| FREE FERULIC ACID [ppm] | HPLC | 3.43 |

### Addition of Water:

5 hl water was added at 2° C.

17 hl water was reached and temperature decreased to 57° C.

Sampling:

| | | |
|---|---|---|
| TOTAL SOL. PENTOSANS [mg/ml] | phloroglucinol | 2.083 |
| including: | | |
| XYLOSE [mg/ml] | enzyme kit | 0.025 |
| ARABINOSE [mg/ml] | enzyme kit | 0.257 |
| FREE FERULI ACID [ppm] | HPLC | 1.86 |

2nd Day—Brewing: Mashing-Boiling-Fermentation

### Mashing:

Initial temperature: <45° C.:

Sampling:

| | | |
|---|---|---|
| TOTAL SOL. PENTOSANS [mg/ml] | phloroglucinol | 2.170 |
| including: | | |
| XYLOSE [mg/ml] | enzyme kit | 0.102 |
| ARABINOSE [mg/ml] | enzyme kit | 0.317 |
| FREE FERULIC ACID [ppm] | HPLC | 2.18 |

300 kg ground malt (17.6 wt %) is added to the 17 hl mixture

The mashing process is begun:

1st step: 7 minutes at 45-50° C.

2nd step: at 60-62° C.

3rd step (called “saccharification”): at 66-68° C.

4° step: at 78° C.

Approximately 190 Brix is reached

### Filtration:

Wort was filtered and separated from spent grain. Spent grain was washed twice with 10 hl water at 78° C.: wort was diluted to 27 hl and degrees Brix dropped from 19 to 11.

### Boiling:

At this point wort was boiled for approximately two hours and hops was added, then separated by centrifugation

### Fermentation:

Fermentation occurred at 5 to 8° C. as a result of the action of the Saccharomyces Cerevisiae.

### Precipitation of the Yeast:

After fermentation, temperature was taken to 0° C.: yeast precipitated and was separated.

Beer was left in the tank another 4 weeks and then bottled without filtration.

### Final Yield:

25 hl of low GI beer was obtained.

### Example 3

### Preparation of a Phytocomplex

Preparation of the Starter

Preparation of 350 ml hydrolysate from Example 1, 150 Brix at pH 4.2, microbiologically and enzymatically stabilised, under mild agitation for 2 hours.

Inoculation with Komagataeibacter xylinus cells.

Incubation at a temperature of 28° C. in controlled aerobic conditions for 150 hours until 7° Brix and pH 3.9 were reached and continuous observation of the optical density.

Assessment of kinetics and of concentrations of organic acids.

Preparation of the Fermentation Broth

Preparation of 70 1 of apple preparation at 15° Brix and pH 3.5, microbiologically and enzymatically stabilised, under mild agitation for 2 hours.

Inoculation with the Starter

Inoculation with the starter at a temperature of 28° C. in controlled aerobic conditions for 140 hours until 7° Brix and pH 3 were reached and continuous observation of the optical density. Assessment of kinetics and of concentrations of organic acids; expected cell concentration: >10⁴

Lowering of the temperature to T=12-14° C. under mild agitation with controlled reduction.

Drain and transfer to containers.

### Example 4

### Measurement of the Glycaemic Index of a Beer Obtained According to the Present Invention

The following study was performed to measure the glycaemic index of the beer obtained according to Example 2.

In particular, the aim of this study was to measure the effect on the glycaemic curve resulting from the addition of the hydrolysate according to the invention to a beer. On 10 healthy volunteers, an original beer as such and the same beer, but obtained as in Example 2, i.e. including the addition of the hydrolysate of the invention (shortly “JAX+ beer”), were tested and compared to each other.

The two beers compared had the following characteristics:

| | Original beer | JAX + beer (Ex. 2) |
|---|---|---|
| total arabinoxylans (mg/ml) | 1.230 | 2.640 |
| xylose (mg/ml) | 0.050 | 0.220 |
| arabinose (mg/ml) | 0.053 | 0.170 |
| glucose (mg/ml) | 0.086 | 0.095 |
| free ferulic acid (ppm) | — | 0.340 |
| beta-glucans (ppm) | 151.10 | 213.50 |

From the table above, it follows that, through the process of the invention, additional 1.41 mg/ml (0.14%) of Arabinoxylans (Ax) were solubilised (further to those naturally present), having the following molecular weights:

| | | |
|---|---|---|
| >100 | kDa | 7% |
| 50-100 | kDa | 11% |
| 30-50 | kDa | 12% |
| 10-30 | kDa | 9% |
| 5-10 | kDa | 30% |
| <5 | kDa | 30% |

The method adopted to measure the glycaemic index is scientifically recommended and standardised according to a scientifically validated protocol (Nutrition Subcommittee of the Diabetes Care Advisory Committee of Diabetes UK. The implementation of nutritional advice for people with diabetes. Diabetic Med 2003; 20:786-807). The method took into account the recommendations of Wolever et al. (Canadian Diabetes Association Clinical Practice Guidelines Expert Committee. Canadian Diabetes Association 2003 clinical practice guidelines for the prevention and management of diabetes in Canada: nutrition therapy. Can J Diabetes 2003; 27 (suppl 2): S27-31). The two samples of beer to be tested were administered to 10 volunteers on different days and using a double-blind approach. A 333 ml bottle of beer was provided. The product was administered in the morning at around 9.00 a.m., after a night-time fast of approximately 10-12 hours. For each test the fasting glycaemia level was measured and then the glycemia level was measured every 15 minutes after consumption of the beer (15, 30, 45, 60, 90, 120 after consumption of the beer) according to the protocol reported by Wolever et al. During the trial, the only beverage the subjects could drink was water. For the determination of the glycaemia, full blood sampling was performed by means of capillary testing (finger) using OneTouch Ultra Easy manufactured by LifeScan, which provides the glucose value expressed as mg/100 ml.

The data obtained following administration of the original beer and JAX+ beer were analysed for each subject and the area under the curve (AUC) of the glycaemic response was calculated using ORIGIN mathematical and statistical analysis software.

Subsequently, the ratio was determined between the AUC obtained from the measurement of the JAX+ beer (AUC2) and the area under the curve obtained from the administration of the original beer (AUC1).

Since it was not possible to administer an amount of beer containing 50 g glucose (amount equivalent to 2.94 litres of beer) to the subjects, the effect of the addition of JAX+ on the original beer was assessed by comparison with the original beer.

| | Original beer | JAX + beer | AUC2/AUC1 (%) |
|---|---|---|---|
| AUC | 32.85 ± 2.55 | 19.73 ± 3.06 | 58.05 ± 7.06 |
| t-test | P < 0.002 | | |

The study showed, therefore, a marked hypoglycaemia-inducing effect of the JAX+ beer, which determined a 42% reduction in the glycaemic response compared to the original beer and the reduction was statistically significant (P<0.02). The hypoglycaemic-inducing effect was measured through the study of the dynamics of the glycaemic response in the two hours following administration of the beverage.

### Example 5

### Measurement of the Glycaemic Index of a Fruit Juice Obtained According to the Present Invention

The aim of this study was to measure the effect on the glycaemic curve of the addition of a dry hydrolysate obtained according to the Example 1A (referred to for brevity as “JAX+”) to a commercially available fruit juice. 10 healthy volunteers were enrolled to test a glucose bolus, an original fruit juice, and the fruit juice supplemented with JAX+; these volunteers did not suffer from glycaemia-related disorders and had no familiarity with diabetes. The JAX+ dry hydrolysate was added to the original commercially available fruit juice at the time of testing, with the volunteers advised to shake the beverage well, before consuming it.

The nutritional composition of the original commercially available fruit juice under exam was:
fruit content: 99.8%;
Ingredients: apple juice, sour cherry, elderberry, and blackcurrant, apple puree, raspberry puree, magnesium carbonate, cinnamon extract (0.07%), hemp seed extract (0.05%), vitamin C;
nutritional values for 100 ml:

| Energy | 196 kJ (46k cal) | |
|---|---|---|
| Fats | <0.5 | g |
| including saturated fatty acids | <0.1 | g |
| Carbohydrates | 10 | g |
| including sugars | 10 | g |
| Proteins | <0.5 | g |
| Salt | <0.01 | g |

| | |
|---|---|
| Magnesium | 28 mg = 7.5% VNR |
| Vitamin C | 24 mg = 30% VNR |

Therefore, 500 ml of the original fruit juice used (i.e. one carton of product) contains 50 g carbohydrates, allowing the administration to each volunteer of an identical amount to that of the glucose bolus consisting of 50 g pure glucose.

The fruit juice was supplemented with 4 g/l dry hydrolysate JAX+ comprising the following components:

| | |
|---|---|
| Total arabinoxylans | 50.00% |
| Xylose | 1.16% |
| Arabinose | 0.32% |
| Glucose | 0.00% |

The blood samples were taken intravenously at the Centro Diagnostico Italiano (Milan), which provided the over-time glycaemia and insulinemia values for subsequent data analysis.

The method adopted to measure the glycaemic index is scientifically recommended and standardised according to a scientifically validated protocol (Nutrition Subcommittee of the Diabetes Care Advisory Committee of Diabetes UK. The implementation of nutritional advice for people with diabetes. Diabetic Med 2003; 20:786-807). The method took into account the recommendations of Wolever et al. (Canadian Diabetes Association Clinical Practice Guidelines Expert Committee. Canadian Diabetes Association 2003 clinical practice guidelines for the prevention and management of diabetes in Canada: nutrition therapy. Can J Diabetes 2003; 27 (suppl 2): S27-31).

The data relating to insulinemia were analysed with the same mathematical procedure.

The glucose bolus, and the two samples of fruit juice to be tested were administered to 10 volunteers on different days, following a double-blind approach.

The 3 samples were administered in the morning, at approximately 9.00 a.m., after night-time fast lasting 10-12 hours. For each test, the fasting glycaemia measurement was taken and, subsequently, every 15 minutes after the beverage was consumed (15, 30, 45, 60, 90, 120 minutes), according to the protocol reported by Wolever et al.

During the trial, the only beverage the subjects could drink was water.

The data obtained following administration of the glucose bolus, the original juice, and the juice with JAX+ were analysed for each subject. More specifically, for each volunteer the area under the curve (AUC2) of the glycaemic response was calculated using ORIGIN mathematical and statistical analysis software. Subsequently, the ratio was determined between the area under the curve of the beverage (AUC1) and the area under the curve obtained from the administration of the glucose standard (AUC2).

This ratio (AUC1/AUC2) gives the glycaemic index of the beverage in question of the individual volunteers. Finally, for each of the beverages the following values were assessed: the mean GI, the standard deviation (SD) and the mean squared error (MSE). A low glycaemic index product is defined as such when derived from a ratio (AUC1/AUC2) equal to or less than 55.

The analysis of the glycaemia thus performed gave the following results:

| | Original juice | Juice with JAX + | |
|---|---|---|---|
| | (mean ± MSE) | (mean ± MSE) | Reduction % |
| Glycaemic index (GI) | 52.35 ± 5.5 | 40.5 ± 7.3 | −22.6 % |
| t-test | P < 0.002 | | |

The value obtained shows how the glycaemic response of the juice with JAX+ is 22.6% lower than that of the original juice available on the market. The students' t-test used for the statistical analysis shows how the result obtained is statistically significant (P<0.02).

The analysis of the insulinemia thus performed has given the following results:

| | Original juice | Juice with JAX + | |
|---|---|---|---|
| | (mean ± MSE) | (mean ± MSE) | Increase % |
| Insulinemic index | 47.8 ± 7.7 | 57.8 ± 4.3 | +19.7 % |
| t-test | P < 0.01 | | |

It should be noted that the addition of JAX+ induces a 19.7% increase in the insulinemic area of the juice. In particular, the dynamic analysis of the insulinemic curve of the different subjects 30 minutes after the consumption of the fruit juice with JAX+ shows an extremely significant increase in the insulin which slows, delays, or prevents the reactive hypoglycaemia present in the subjects after consumption of the juice.

In conclusion, the study shows a hypoglycaemia-inducing effect of the fruit juice with JAX+ which determines a 22% reduction in the glycaemic response and a parallel 19.7% increase in insulinemia. Furthermore, in the 70% of the volunteers, the glycaemic response of the fruit juice with JAX+ prevented the reactive hypoglycaemia which is characteristic of the original juice available on the market and characteristic of high glycaemic index foods.

### Example 6

### Measurement of the Glycaemic Index of a Chocolate Obtained According to the Present Invention

The aim of this study was to measure the glycaemic index of 2 types of dark chocolate. The method adopted to measure the glycaemic index is scientifically recommended and standardised according to a scientifically validated protocol (Nutrition Subcommittee of the Diabetes Care Advisory Committee of Diabetes UK. The implementation of nutritional advice for people with diabetes. Diabetic Med 2003; 20:786-807). The method took into account the recommendations of Wolever et al. (Canadian Diabetes Association Clinical Practice Guidelines Expert Committee. Canadian Diabetes Association 2003 clinical practice guidelines for the prevention and management of diabetes in Canada: nutrition therapy. Can J Diabetes 2003; 27 (suppl 2): S27-31).

The reference value used was a solution containing 50 g of glucose for which the ratio with the food to be tested was established mathematically. The samples in question were administered to 10 volunteers, in portions providing 50 g available carbohydrates, defined as total carbohydrates net of fibre. The products were administered in the morning at approximately 9.00 a.m. on different days, after night-time fasting lasting 10-12 hours. For each test, the fasting glycaemia measurement was taken and, subsequently, every 15 minutes after the food was consumed (15, 30, 45, 60, 90, 120 minutes) according to the protocol reported by Wolever et al. During the meal, the only beverage the subjects could drink was water. For the determination of the glycaemia, full blood sampling was performed by means of capillary testing (finger) using OneTouch Ultra Easy manufactured by LifeScan, which provides the glucose value expressed as mg/100 ml.

For each volunteer, after administration of the glucose bolus and after each food to be tested, the area under the curve (AUC1) of the glycaemic response was calculated using ORIGIN mathematical and statistical analysis software. Subsequently, the ratio was determined between the area under the curve AUC1 and the area under the curve obtained from the administration of the glucose standard (AUC2).

This ratio (AUC1/AUC2) gives the glycaemic index of the food in question of the individual volunteers. Finally, for each of the food items, the following values were assessed: the mean GI, the standard deviation (SD) and the mean squared error (MSE).

The two types of dark chocolate tested were:
1) chocolate available on the market (with sugar added),
2) chocolate 1) supplemented with dry hydrolysate comprising 20% arabinoxylans (hydrolysate obtainable from the process in Example 1b).

Shown below are the nutritional values (mean values for 100 g product) of the chocolate available on the market 1):

chocolate available on the market

| | |
|---|---|
| 9.54 | Proteins (g) |
| 53.86 | Total lipids (g) including |
| 17.07 | Monounsaturated fatty acids (g) |
| 2.85 | Polyunsaturated fatty acids (g) |
| 33.42 | Saturated fatty acids (g) |
| 0.00 | Trans fatty acids (g) |
| 0.00 | Cholesterol (mg) |
| 21.35 | Carbohydrates (g) including |
| 11.20 | sugars (g) |
| 3.36 | starch (g) |
| 8.14 | Sodium (mg) |
| 728.20 | Potassium (mg) |
| 0.00 | Iron (mg) |
| 0.00 | Calcium (mg) |
| 12.79 | Fibre (g) |
| 0.00 | vit. A (μg) |
| 0.00 | vit. C (mg) |
| 0.00 | vit. E (mg) |
| 0.000000 | vit. D (μg) |
| 0.00000 | Organic acid |
| 0.020 | Salt (g) |

The glycaemia test thus conducted gave the following results:

| | Glycaemic index (GI) |
|---|---|
| | (mean ± MSE) |
| 1) chocolate available on the market | 98.34 ± 8.93 |
| 2) chocolate 1) supplemented with | 35.45 ± 3.28 |
| dry hydrolysate | |

As said, a low glycaemic index food is a product with a (AUC1/AUC2) ratio giving a value equal to or less than 55.

From the above test, it clearly emerged that the chocolate comprising the hydrolysate of the present invention, is not merely a low glycaemic index product, but rather an extremely low glycaemic index product, since it has a glycaemic index which is approximately 64% of that of the chocolate available on the market, with the same amount of added sugars.

1. A process for the preparation of a brewer's spent grain hydrolysate, said process comprising the steps of:
i) mixing brewer's spent grain and water, in a weight ratio of 1:1 to 1:5,
ii) adding to the mixture thus obtained up to 2 wt %, based on the weight of the mixture, of an enzyme consisting of:
a) xylanase, or
b) an enzymatic complex of xylanase, amylase and glucanase,
and letting to react for 1-6 hours at a temperature of 45-65° C. and pH of 4-6,
iii) deactivating the enzyme of step ii), by increasing the temperature to 80-90° C. for at least 5 minutes, and
iv) separating the liquid component from the solid component obtained at the end of step iii), thus keeping the liquid component, which is the brewer's spent grain hydrolysate.

2. The process of claim 1, wherein said xylanase is endo-1,4-beta-xylanase, said amylase is alpha-amylase and said glucanase is endo-1,3(4)-beta-glucanase.

3. The process of claim 1, wherein, in step ii), the enzyme is added in an amount up to 1 wt %, based on the weight of the mixture.

4. The process of claim 1, wherein, in step i), brewer's spent grain and water are in weight ratio of 1:1.5 to 1:3.

5. The process of claim 1, wherein, in step ii), the mixture is let to react for 2-4 hours at 58-62° C.

6. The process of claim 1, wherein, in step iv), the liquid component is separated from the solid component by filtration.

7. A brewer's spent grain hydrolysate obtainable by the process of claim 1, wherein in step ii) the enzyme xylanase a) is added, said hydrolysate comprising ferulic acid, proteins, up to 30 mg of beta-glucans and up to 300 mg of pentosans having a number average molecular weight not higher than 30 kDa, per gram of dried hydrolysate.

8. The brewer's spent grain hydrolysate of claim 7, comprising up to 25 mg of beta-glucans and up to 250 mg of pentosanes having a number average molecular weight not higher than 30 kDa, per gram of dried hydrolysate.

9. The brewer's spent grain hydrolysate of claim 7, comprising up to 0.5 mg of ferulic acid, per gram of dried hydrolysate.

10. The brewer's spent grain hydrolysate of claim 7, comprising up to 8 mg of proteins, per gram of dried hydrolysate.

11. A brewer's spent grain hydrolysate obtainable by the process of claim 1 wherein in step ii) the enzymatic complex b) of xylanase, amylase and glucanase is added, said hydrolysate comprising ferulic acid, proteins, traces of beta-glucans and up to 250 mg of pentosans having a number average molecular weight not higher than 10 kDa, per gram of dried hydrolysate.

12. The brewer's spent grain hydrolysate of claim 11, comprising up to 200 mg of pentosanes having a number average molecular weight not higher than 5 kDa, per gram of dried hydrolysate.

13. The brewer's spent grain hydrolysate of claim 11, comprising up to 0.8 mg of ferulic acid, per gram of dried hydrolysate.

14. The brewer's spent grain hydrolysate of claim 11, comprising up to 5 mg of protein, per gram of dried hydrolysate.

15.-16. (canceled)

17. A food supplement, or a food product, or a medical device or a phytocomplex, for human and animal, comprising the brewer's spent grain hydrolysate of claim 7, or the brewer's spent grain hydrolysate of claim 11.

18. A brewing process, comprising the steps of:
1) adding water and the brewer's spent grain hydrolysate obtained from step iv) of the process of claim 1, in a mashing tun,
2) adding ground malt, thus proceeding to the mashing,
3) filtering the wort resulting from step 2),
4) boiling the wort and adding hops,
5) separating the wort from hops and fermenting in the presence of yeasts,
6) separating the yeasts and solid residues from the beer thus obtained, and optionally
7) letting the beer to age.

19.-21. (canceled)
